(19) United States
(12) Patent Application Publication
(10) Pub. No.: US 2011/0270285 A1
(43) Pub. Date: Nov. 3, 2011

(54) DISPOSITION INTRODUCED TO A SURGICAL CLIP

(76) Inventor: Fernando César Toniazzi Lissa, Criciuma (BR)

(21) Appl. No.: 12/838,819

(22) Filed: Jul. 19, 2010

(30) Foreign Application Priority Data
Jul. 23, 2009 (BR) MU8901448-0

(51) Int. Cl. A61B 17/08

(52) U.S. Cl. 606/151

(57) ABSTRACT

DISPOSITION INTRODUCED TO A SURGICAL CLIP refers to a clip formed by two long leg members, joined by two converging portions of articulation that may or may not have narrowing portions that facilitates folding. The inner surface of said leg members are provided with a series of pyramid-shaped grooves, arranged in an alternate and staggered way in two parallel lines, acting as fixation teeth when the clip is closed, while the reverse surface of said legs presents longitudinal channels. Alternatively, the grooves can be arranged in continuous lines positioned at different levels in each leg. The grooves may also present a parallelepipedic shape and being arranged in two alternating and staggered parallel lines. It is also an alternative that the parallelepipedic shaped grooves extend vertically from top to bottom of the legs and are arranged in positions that do not coincide.

### CROSS-REFERENCE TO RELATED APPLICATION

This application claims the priority filing date of Brazilian patent application no. MU8901448-0 filed in Brazil on Jul. 23, 2009.

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The present utility model belongs, in a general way, to the technical field of surgical tools and equipments and refers, more specifically, to a new constructive disposition applied to a clip for occlusion of vascular or intestinal tubular structures (as the cecal appendix), and was developed specifically to perform the occlusion of vessels of greater caliber and/or greater pressure than the smaller ones that could be operated using conventional surgical clips.

There are several situations in surgical procedures in which it is necessary to limit the blood flux in tubular structures, such as blood vessels, to keep the surgical field free of excess blood, to close or to link several vessels together before severance of the same, to reduce blood loss of the patient, to remove tumors and so forth.

The state of art of this technical field comprise surgical hemostatic clips to tubular structures that are deformed and stapled over said vessels, obtaining the occlusion of the same. These conventional clips however are efficient only at vessels of low caliber and at low pressures.

Examples of surgical clips of this type are described in the Brazilian patent applications BRPI90002402, “hemostatic staple”, which describes a hemostatic clip comprising two legs joined by an articulated region, with one leg provided with a longitudinal groove on the surface of contact with the tissue and the other equipped with a longitudinal recess aligned and coincident with said slot so as to facilitate the introduction of the slot in the recess when the clip is closed, providing a further series of transverse grooves and recesses to optimize fixation; BRPI 8801652-8, “Hemostatic surgical clip,” which describes a clamp designed to solve the problem of spacing or uneven pressure closing the clamp, using both for a crack or depression disposed on the outer contact surface of the device for applying the clip, which promotes a tendency for the legs not to bend to a substantially straight condition; BRPI8300718-0, “Hemostatic clamp,” that describes a staple manufactured with biocompatible polymeric materials, which comprises two leg members connected to the proximal ends by a section of elastic joint and movable locking means; BRPI8302105-1, “Hemostatic clamp,” which describes clips manufactured from non-deformable and substantially non elastic polymeric materials, comprising a pair of leg members fixed or detachable at the proximal ends, which differ from one another when the clamp is in the open position and are substantially parallel when the clamp is in the closed position.

All the above cited devices are not effective in the occlusion of structures of greater sizes or pressure, which limits the application of the same and make clear the need to develop a clip that can effectively be used in such structures.

This utility model aims to characterize a surgical clip or staple that can be used in tubular structures coarser, with larger size and/or pressure (vascular structures and/or bowel), with higher accuracy, greater safety and greater effectiveness.

These objectives will be achieved through a new constructive disposition that is more robust and more resistant. The surgical clip is equipped with slots that act as teeth, gripping the tubular structure more efficiently and not letting go despite the forces applied against it, effectively keeping the clip stationary. These slots allow for a better grip and setting, and cause a safer occlusion.

### SUMMARY

A disposition introduced to a surgical clip, in particular, a clip formed by two long leg members, joined by two converging portions of articulation that may or may not have narrowing portions that facilitates folding. The inner surface of said leg members are provided with a series of pyramid-shaped grooves, arranged in an alternate and staggered way in two parallel lines, acting as fixation teeth when the clip is closed, while the reverse surface of said legs presents longitudinal channels. Alternatively, the grooves can be arranged in continuous lines positioned at different levels in each leg. The grooves may also present a parallelepipedic shape and being arranged in two alternating and staggered parallel lines. It is also an alternative that the parallelepipedic shaped grooves extend vertically from top to bottom of the legs and are arranged in positions that do not coincide.

### DRAWINGS

FIG. 1 is a top plan view of the surgical clip.

FIG. 2 is a cross sectional view AA referenced at the FIG. 1.

FIG. 3 is the plan view BB indicated at the FIG. 2.

FIG. 4 is a cross sectional view C referenced at the FIG. 1.

FIG. 5 is a top plan view of another embodiment of the surgical clip.

FIG. 6 is the plan view AA referenced at the FIG. 5.

FIG. 7 is the plan view BB referenced at the FIG. 5.

FIG. 8 is a cross sectional view CC referenced at the FIG. 5.

FIG. 9 is a top plan view of another embodiment of the surgical clip.

FIG. 10 is the plan view AA referenced at the FIG. 9.

FIG. 11 is the plan view BB referenced at the FIG. 9.

FIG. 12 is a cross sectional view CC referenced at the FIG. 9.

FIG. 13 is a top plan view of another embodiment of the surgical clip.

FIG. 14 is the plan view AA referenced at the FIG. 13.

FIG. 15 is the plan view BB referenced at the FIG. 13.

FIG. 16 is a cross sectional view CC referenced at the FIG. 13.

FIG. 17 is a top plan view of another embodiment of the surgical clip.

FIG. 18 is the plan view AA referenced at the FIG. 17.

FIG. 19 is the plan view BB referenced at the FIG. 17.

FIG. 20 is a cross sectional view CC referenced at the FIG. 17.

FIG. 21 is a top plan view of another embodiment of the surgical clip.

FIG. 22 is the plan view AA referenced at the FIG. 21.

FIG. 23 is the plan view BB referenced at the FIG. 21.

FIG. 24 is a cross sectional view CC referenced at the FIG. 21.

FIG. 25 is a top plan view of another embodiment of the surgical clip.

FIG. 26 is the plan view AA referenced at the FIG. 25.

FIG. 27 is the plan view BB referenced at the FIG. 25.

FIG. 28 is a cross sectional view CC referenced at the FIG. 25.

FIG. 29 is a top plan view of another embodiment of the surgical clip.

FIG. 30 is the plan view AA referenced at the FIG. 29.

FIG. 31 is the plan view BB referenced at the FIG. 29.

FIG. 32 is a cross sectional view CC referenced at the FIG. 29.

### DETAILED DESCRIPTION OF THE DRAWINGS

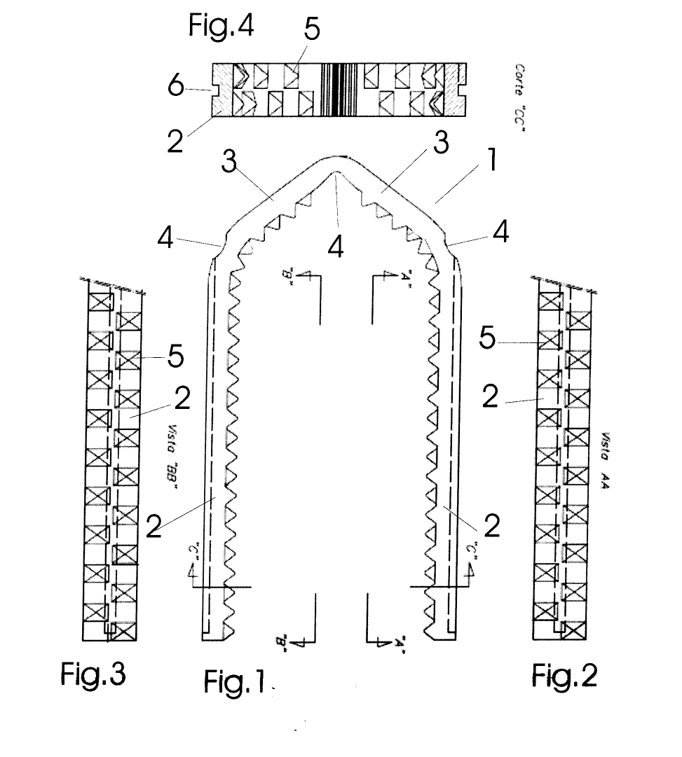

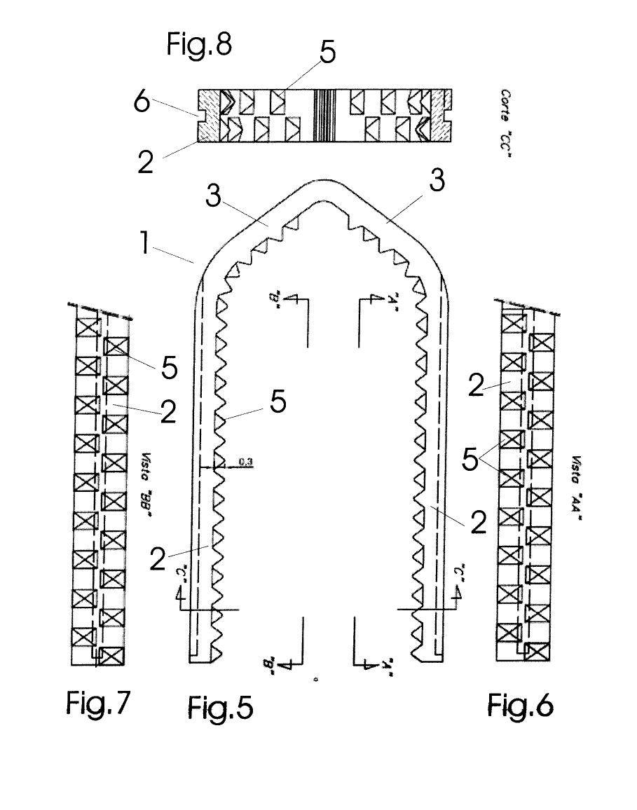

As we can see at the FIGS. 1 to 4, the disposition introduced to a surgical clip refers to a clip (1) formed by two long leg members (2), joined by two converging portions of articulation (3) that can have (as shown in FIGS. 1 to 4) or may not have (as shown in FIGS. 5 to 8) narrowing portions (4) that facilitates folding. The inner surface of said leg members (2) are provided with a series of pyramid-shaped grooves (5), arranged in an alternate and staggered way in two parallel lines, acting as fixation teeth when the clip (1) is closed, while the reverse surface of said legs (2) presents longitudinal channels (6).

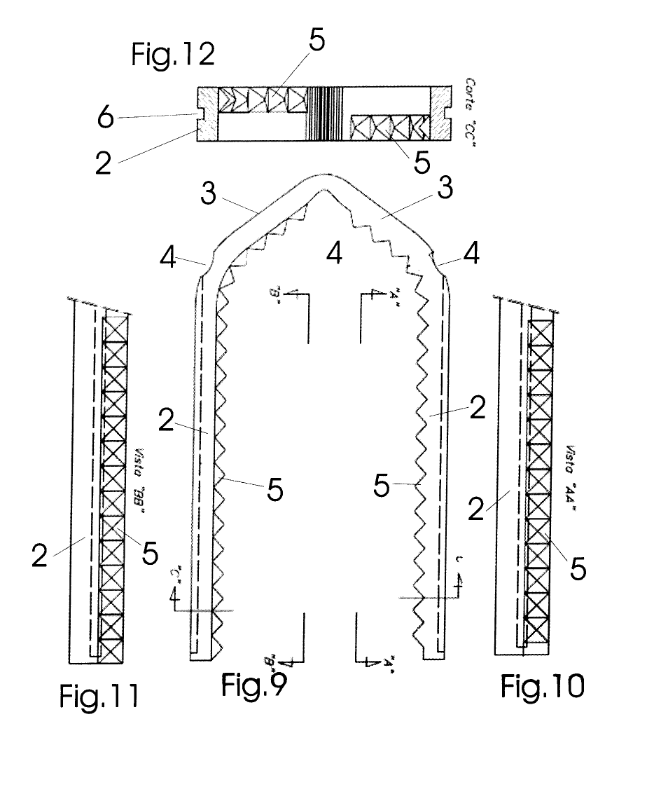

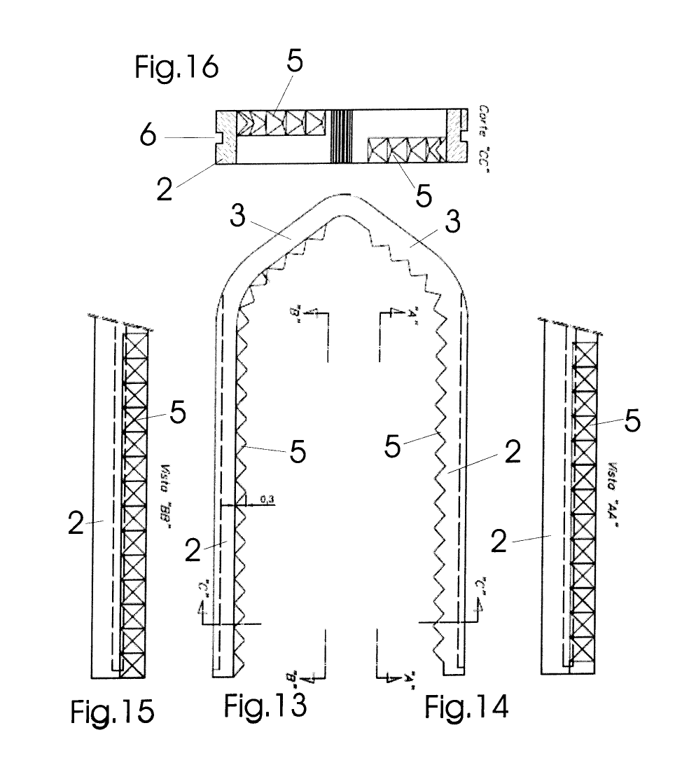

Alternatively, the grooves (5) can be arranged in continuous lines (without the spaces in the sequence), in which said lines are positioned at different levels in each leg (2) (as shown in FIGS. 9 to 12) with or without (as shown in FIGS. 13 to 16) the narrowing portions (4).

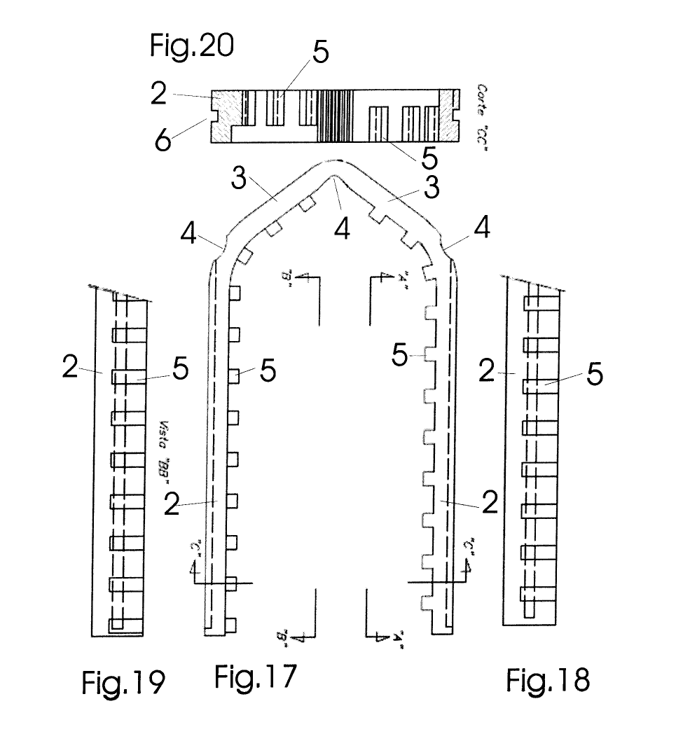

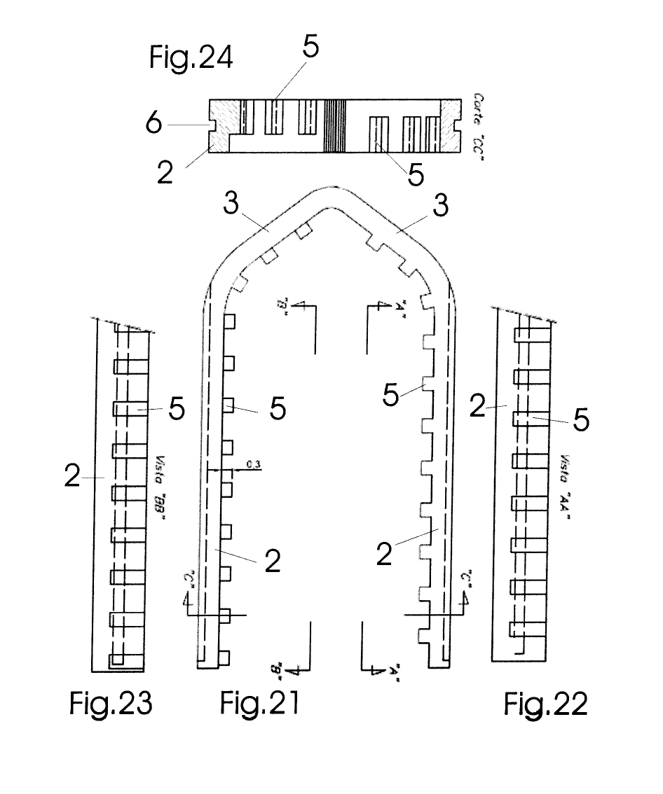

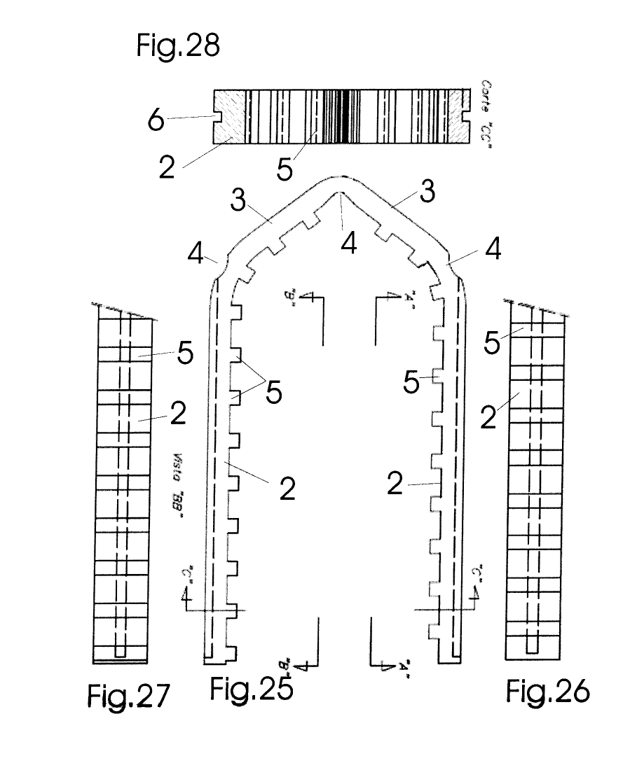

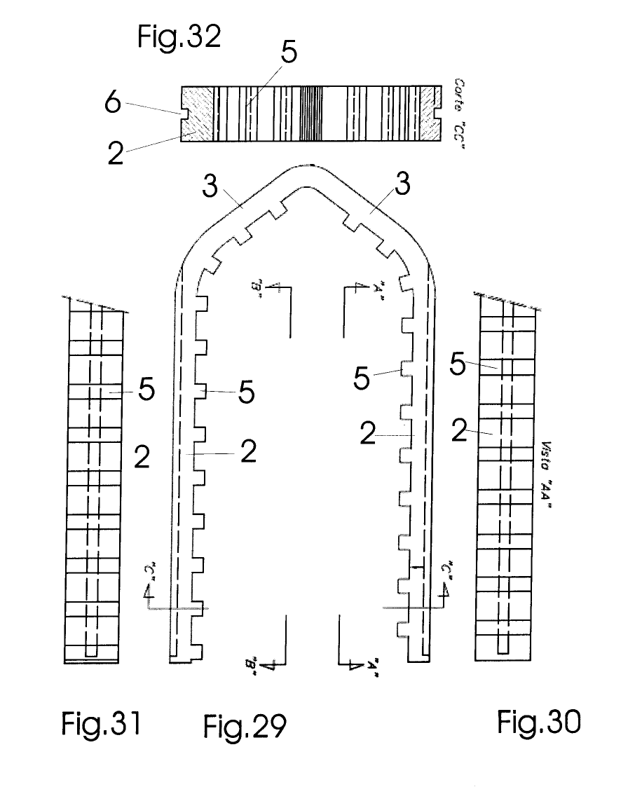

The grooves (5) may also present a parallelepipedic shape and being arranged in two alternating and staggered parallel lines, as well as being provided (as shown in FIGS. 17 to 20) or deprived (as shown in FIGS. 21 to 24) from the narrowing portions (4). It is also an alternative that the parallelepipedic shaped grooves (5) extend vertically from top to bottom of the legs (2) and are arranged in positions that do not coincide, so each groove (5) of each leg (2) can be received in the empty space between two consecutive grooves (5) of the other leg (2).

All features disclosed in this specification, including any accompanying claims, abstract, and drawings, may be replaced by alternative features serving the same, equivalent or similar purpose, unless expressly stated otherwise. Thus, unless expressly stated otherwise, each feature disclosed is one example only of a generic series of equivalent or similar features.

Although preferred embodiments of the present invention have been shown and described, various modifications and substitutions may be made thereto without departing from the spirit and scope of the invention. Accordingly, it is to be understood that the present invention has been described by way of illustration and not limitation.

1. DISPOSITION INTRODUCED TO A SURGICAL CLIP characterized as being formed by two long leg members (2), joined by two converging portions of articulation (3) that may or may not present narrowing portions (4) that facilitates folding, presenting a series of pyramid-shaped grooves (5), arranged in an alternate and staggered way in two parallel lines over the inner surface of said leg members (2) acting as fixation teeth when the clip (1) is closed, while the reverse surface of said legs (2) presents longitudinal channels (6).

2. The DISPOSITION INTRODUCED TO A SURGICAL CLIP as claimed in 1 and additionally characterized by pyramid-shaped grooves (5) disposed in continuous lines positioned at different levels in each leg (2).

3. The DISPOSITION INTRODUCED TO A SURGICAL CLIP as claimed in 1 and additionally characterized by the grooves (5) presenting a parallelepipedic shape and being arranged in two alternating and staggered parallel lines.

4. The DISPOSITION INTRODUCED TO A SURGICAL CLIP as claimed in 3 and additionally characterized by the grooves (5) extending vertically from top to bottom of the legs (2) and arranged in positions that do not coincide.
